(19) United States
(12) Patent Application Publication
(10) Pub. No.: US 2011/0027207 A1
(43) Pub. Date: Feb. 3, 2011

(54) EYELASH AND EYEBROW FORTIFIER

(76) Inventor: Ben Kaminsky, Montreal (CA)

Correspondence Address: BAKER & RANNELLS, PA, 575 ROUTE 28, SUITE 102, RARITAN, NJ, 08869, US

(21) Appl. No.: 12/510,329

(22) Filed: Jul. 28, 2009

(51) Int. Cl. A61K 8/73; A61Q 5/00

(52) U.S. Cl. 424/70.1

(57) ABSTRACT

The present invention describes a composition containing six families of compounds with physiologically active properties for enhancing and maintaining full, long and healthy eyelashes and eyebrows. The compounds include amino acids, peptides, saccharides, alpha hydroxy acids, phenolic compounds, organic or inorganic minerals, and plant based hormones. The unique combination of these ingredients provides for enhanced appearance and growth and meeting proper nutritional dermal requirements for eyelashes and eyebrows.

### FIELD OF THE INVENTION

This invention describes the use of a complex physiological active humectant for use in eyelash and eyebrow fortifying formulations which provide enhanced appearance and growth of the eyelashes and eyebrows, moisturizing ingredients and enriching compounds that will stimulate healthy skin and hair. This fortifier composition has been dermatologist tested and found to be hypoallergenic and gentle to the skin, containing many natural compounds and derivatives, which allows for a wide range of formulation options with ingredients that may be typically used in eyelash and eyebrow cosmetics.

### BACKGROUND OF THE INVENTION

Formulation for coloring, styling and curling eyelashes and eyebrows have been commercially available for many years and include the typical ingredients that can be found in hair and skin care type products. These include polymers for styling aids which provide longer looking eyelashes. Coloring agents are added to give the eyelashes and eyebrows a dark color and more substance. Cationic ingredients are added to provide better compatibility of the formulation to the eyelashes and eyebrows. Proteins and amino acids are added too stimulate healthy skin and lash formation and growth.

Eyelashes and eyebrows are a natural hair like material consisting primarily of keratinous (protein) fibers. The art of coloring and styling the eyelashes and eyebrows has been practiced for years, especially in women's fashion, and has developed into a very large business opportunity.

New trends in cosmetics include all natural, green, and more gentle ingredients. The formulations described in the application are derived from natural sources and thus make them more environmentally friendly and have better acceptability by sensitive skin.

### DETAILED DESCRIPTION OF THE INVENTION

The physiological active humectant of this invention is defined as containing 6 various types of compounds and in specific ranges to optimize their effectiveness. The first class of materials in the physiological humectant is the class of natural derived saccharides. These include simple sugars such as glucose, fructose, galactose, glucuronic acid and similar mono- and di-pentoses and hexoses. Also in this portion of the mix is a blend of oligosaccharides and polysaccharides such as starch (both amylose and amylopectin) and cellulosic compounds. All the materials in this fraction of the formulation must be water soluble to be effective in the formulation. The total percent of these saccharides in the overall weight of the physiological active humectant is from about 90 to about 100 percent. In one embodiment of this invention, the percent of the saccharides the humectant are between from about 90 to about 96 percent of the total dry weight.

A second family of ingredients in the humectant are amino acids and low molecular weight polypeptides (proteins). In one embodiment of this invention the amino acids and low molecular weight polypeptides are obtained from plant sources. In another embodiment of this invention the amino acids and peptides are present from about 0.1 to about 5.0 percent of the total dry weight of the physiological active humectant.

A third family of the compound present in the humectant are phenolic and polyphenolic compounds. These compounds are often highly colored materials which act as very strong anti-oxidants in fruits and vegetables. This type of anti-oxidant is found in blue-berries, grapes, cranberries, rhubarb and the like. These phenolic compounds are always isolated from plant sources and are present in the humectant formulation from about 0.01 to about 0.5 percent of the total dry humectant weight.

The fourth family of compounds in the physiological humectant is again isolated from many different plant sources and is broadly classified as alpha hydroxy carboxylic acids. Some non limiting examples of these acids are lactic acid and malic acid. In one embodiment of his invention the alpha hydroxy acid is malic acid. The alpha hydroxy acid is present in the humectant at from about 0.5 to about 2.5 percent of the total dry humectant weight.

Yet another important piece of the humectant formulation is classified as minerals. The include calcium, zinc, potassium, manganese and phosphorus. These minerals can be in many different forms such as phosphates, phosphonates and various organic phosphorus containing compounds. Some non-limiting examples of organic minerals are calcium panthotlienate, zinc gluconate, and potassium acetate. The main consideration for these minerals is that they be in a form that is acceptable for application to the hair or skin. In one embodiment of this invention the minerals are provided in the formulation in a highly bioavailable form. In another embodiment of this invention the total amount of minerals are present from about 0.1 to about 0.5 percent of the total dry humectant weight.

The final set of ingredients is those obtained from plants and fall into the general category of vegetable hormones. Non-limiting examples of such compounds are estrogen mimics isolated from soy protein, beta sistosterol a plant based cholesterol type material and other phytosterols. Also included in this class of compounds are cytokinins, abscisic acid and various indole derivatives. These materials are isolated from plant based extracts and are present in the physiological active humectant from about 0.01 to about 0.1 percent of the total humectant weight.

The physiological active humectant is incorporated into the eyelash and eyebrow fortifier formulation at an effective amount to provide beneficial properties to the eyelashes, eyebrows and skin around the eye. As part of the fortifier formulation, the physiological active humectant is present in the fortifier formulation from about 1 to about 30% of the total weight of the fortifier. In one embodiment of this invention the physiological active humectant is present in the fortifier formulation at from about 5 to about 25% of the total fortifier weight.

The six classes of compounds have distinct functions in the fortifier, yet some overlap of function can be found. The mono and polysaccharides are present in the humectant primarily to absorb and hold onto the moisture and provide smooth texture and feel to the eyelashes and eyebrows and surrounding area. Depending on the plant source of the starch, variations in molecular weight and architecture may be realized. The native starches come in two basic varieties, amylose and amylopectin. The amylose is a linear in nature and the amylopectin is highly branched. Generally, native type starches extracted from most plant sources will have from about 15 to about 30 percent amylose as a percentage of the total starch weight.

Other polysaccharides may be present depending on the source of the extraction and the desired effect on the fortifier formulation. Typically polysaccharides found and extracted from plants are pectin, cellulose, alginate and guar. These materials can be used alone or in combination with the starch or each other to obtain a specific desired effect. For example, pectin and alginates are very good thickeners, but not particularly good at film forming. Both hold onto water very effectively and make good humectants, but tend to exhibit sticky properties.

The amino acid and peptides are a required component to the formulation as they provide the basic biding blocks for the human cells to synthesize new keratinous fibers (e.g. hair). While certain amino acids can provide a small amount of water holding capacity, their main function in the formulation is as a source of nutrients. Amino acids and peptides which are rich in sulfur (i.e. cysteine) are particularly useful in these fortifier formulations.

The polyphenolic compounds are extremely good antioxidants and can act to reduce or eliminate the effects of sunlight and oxidative processes. It is thought that anti-oxidants are effective in reducing the aging process, but are added to the physiological humectant to aid in cell growth and prevent cell damage. Of particularly interest are those cells that are responsible for regeneration of the eyelashes and eyebrows.

The alpha hydroxy acids are useful for exfoliating skin, and those of particular interest are those that are gentle enough to be used near the eye. Removal of dead skin cells allows for nutrients and moisture to reach the eyelash and eyebrow producing cells and stimulate new growth. Additionally these acids provide a fair amount of moisturizing and softening of the eyelashes and eyebrows (and thus making the eyelashes and eyebrows less brittle and less prone to breakage).

The minerals provide the eyelashes and eyebrows good substance and stimulate hair follicle growth. Not only do the minerals promote growth of healthy tissue, they provide the cells the ability to fight disease. Most cellular processes require many trace minerals for proper cell growth and maintenance.

Plant hormones have now been found to help stimulate new cell growth and promote new eyelashes and eyebrows. These hormones are widely spread through nature and now we have discovered that these hormones, when used in combination with the proper nutrients and minerals can stimulate eyelash and eyebrow growth to produce thicker, fuller looking lashes. These anti-aging hormones which help regulate cell growth by increasing mitochondrial activity are uniquely combined with conditioning and fortifying nutritional ingredients to produce a novel dermatologist tested fortifying preparation.

In addition to the six classes of compounds discussed above as the required elements to this invention, other traditional eyelash or eyebrow cosmetic ingredients can be added to and used in conjunction with the physiological active humectant. These include, but are not limited to colorants, pigments, preservatives, stabilizers, thickeners, gelling agents, styling aids, retention aids, conditioners, flow aids, cosmetically approved solvents and the like.

The physiologically active humectant can be applied to the eyelash or eyebrow in a liquid from aqueous solution, gel or a paste. In addition, solvents such as isopropyl alcohol or ethyl alcohol can be added to help control viscosity and make the formulation easier to apply. Alternatively, the physiologically active humectant can be applied by dusting a solid onto the eyelashes and eyebrows or applied with a brush. One skilled in the art would recognize the endless possibilities to formulate with such a powerful humectant, all of which are covered under the scope of this invention.

### Experimental

The table below shows a typical formulation that incorporates the physiologically active humectant in the eyelash and eyebrow fortifier application. All percents are based on the total dry solids and account for any moisture that may be present in any particular ingredient.

| | Weight (dry) in |
|---|---|
| Ingredient | formulation |
| Water | 66.75 |
| Physiologically active humectant | 25 |
| Glycerin | 2 |
| Butylene Glycol | 2 |
| Panax Ginseng Root Extract | 0.5 |
| PEG-12 Dimethicone | 0.1 |
| Carbomer | 0.5 |
| Caprylyl glycol | 1.0 |
| Panthenol | 0.1 |
| PPG-26-buteth-26 | 0.1 |
| Glucamine | 0.2 |
| PEG-40 hydrogenated Castor oil | 0.05 |
| Arctium Majus root extract | 0.1 |
| Acetyl Tyrosine | 0.2 |
| 1,2 hexanediol | 0.4 |
| Niacinamide | 0.1 |
| Ornithine HCl | 0.01 |
| Citrulline | 0.01 |
| Glucosamine HCl | 0.05 |
| Polyquaternium-11 | 0.5 |
| Biotin | 0.05 |
| Apigenin | 0.01 |
| Oleanolic acid | 0.01 |
| Biotinoyl Tripeptide-1 | 0.1 |
| Phospholipids | 0.005 |
| Tocopheryl acetate | 0.1 |
| Retinyl Palmitate | 0.001 |
| Ascorbyl Palmitate | 0.05 |
| Disodium EDTA | 0.005 |

The carbomer and polyquaternium-11 are added to the water with agitation until all the polymer is dissolved and uniform. The other ingredients are then added to the formulation and mixed until uniform and all ingredients have been added.

What is claimed is:

1. An eyelash and eyebrow fortifier composition comprising a physiologically active humectant containing at least one ingredient from each of six classes; saccharides and sugars, amino acids and peptides, alpha-hydroxy acids, phenolic and polyphenolic compounds, organic or inorganic minerals and plant based hormones.

2. The physiologically active humectant of claim 1 wherein the saccharides and sugars are a blend of starches and mono and di-sugars.

3. The physiologically active humectant of claim 1 wherein the amino acids comprises arginine.

4. The physiologically active humectant of claim 1 wherein the phenolic and polyphenolic compounds comprise ferulic acid, vanillic acid, coumaric acid, synaptic acid, coniferaldehyde, syringaldehyde, vanillin, syringic acid and coniferol.

5. The physiologically active humectant of claim 1 wherein the minerals are organic minerals.

6. The minerals of claim 5 wherein the minerals comprise zinc, calcium, potassium, and phosphorus.

7. The physiologically active humectant of claim 1 wherein the plant based hormones are cyctokinins, abscisic acid and indole derivatives.

8. The eyelash and eyebrow fortifier of claim 1 wherein the physiologically active humectant is present from about 1 to about 30 percent of the total dry fortifier weight.

9. The eyelash and eyebrow fortifier of claim 8 wherein the physiologically active humectant is present from about 5 to about 25 percent of the total dry fortifier weight.
